(19) United States
(12) Patent Application Publication
(10) Pub. No.: US 2024/0358717 A1
(43) Pub. Date: Oct. 31, 2024

(54) APPLICATION OF B-ECDYSTERONE IN PREPARATION OF MEDICINE FOR URIC ACID-LOWERING AND HYPERURICEMIC KIDNEY INJURY

(71) Applicant: Institute of Chinese Materia Medica, China Academy of Chinese Medical Sciences, Beijing (CN)

(72) Inventors: Jingzhuo Tian, Beijing (CN); Aihua Liang, Beijing (CN); Jing Meng, Beijing (CN)

(21) Appl. No.: 18/646,823

(22) Filed: Apr. 26, 2024

(30) Foreign Application Priority Data
Apr. 27, 2023 (CN) 202310465751.X

(51) Int. Cl. A61K 31/575; A61P 13/02

(52) U.S. Cl. CPC A61K 31/575; A61P 13/02

(57) ABSTRACT

The present disclosure provides the application of β-ecdysterone in preparation of a medicine for uric acid-lowering and/or hyperuricemic kidney injury. β-ecdysterone or/and isomers thereof, or/and pharmaceutically acceptable salts, esters or/and prodrugs thereof are the sole active components in the drug, which further comprises a pharmaceutically acceptable carrier thereof; on the one hand, it has a uric acid-lowering effect on hyperuricemia, and on the other hand, it can reduce blood creatinine and urea nitrogen, reduce urate crystals, and thus improve kidney injury in hyperuricemia; it also has the effect of reducing the degree of kidney tubular dilation and wall thinning, as well as reducing the renal epithelial cell detachment. Compared with the prior art, the active ingredient β-ecdysterone of the present disclosure can not only reduce uric acid but also protect the kidney injury of hyperuricemia, and provides a new use for the preparation of drugs by β-ecdysterone.

### CROSS-REFERENCE TO RELATED APPLICATION

The present application claims priority from Chinese Patent Application No. 202310465751.X filed on Apr. 27, 2023, the contents of which are incorporated herein by reference in their entirety.

### TECHNICAL FIELD

The present disclosure herein belongs to the field of pharmaceutical technology and relates to the application of β-ecdysterone in preparation of a medicine for uric acid-lowering and kidney injury, and specifically relates to the application of β-ecdysterone in preparation of a medicine for uric acid-lowering and hyperuricemic kidney injury thereof.

### BACKGROUND

With the improvement of modern people's living standards, the incidence of hyperuricemia is increasing year by year, and the current incidence of hyperuricemia in China is about 13.3%, involving about 170 million people. Hyperuricemia is a metabolic syndrome in which abnormal purine metabolism leads to an increase in uric acid production and/or a decrease in uric acid excretion resulting in abnormally high blood uric acid levels. When the concentration of uric acid in the body is too high, urate crystals can be precipitated and deposited in the kidneys, joints and other tissues, leading to uric acid nephropathy or gout. Hyperuricemia is also an independent risk factor for chronic kidney disease, diabetes mellitus, hypertension and metabolic syndrome and other diseases, which can increase the risk of death. Therefore, there is a significant clinical need to treat hyperuricemia.

In the treatment of hyperuricemia, the most commonly used drugs in the clinic are allopurinol and febuxostat, which are xanthine oxidase inhibitors that inhibit uric acid synthesis, and probenecid and benzbromarone, which promote uric acid excretion. Despite the efficacy of these drugs, there are different degrees of adverse reactions. Allopurinol can cause severe or even fatal skin damage in some patients; febuxostat has unpredictable cardiovascular risks and can increase the risk of death; and benzbromarone can cause severe hepatotoxicity. The National Medical Products Administration has issued warning information about serious adverse reactions to the above drugs. In addition, hyperuricemia can lead to kidney injury, but the clinical application of the current drug instead of kidney protective effect, some will exacerbate the degree of kidney injury (such as drugs: allopurinol, etc.). For these reasons, there is a high demand for the development of drugs with renoprotective effects for treating hyperuricemia clinically.

β-ecdysterone, also known as ecdysterone, 20-hydroxyecdysone, is a naturally occurring steroidal component that is most abundant in herbaceous plants, predominantly in plants such as Achyranthis Bidentatae Radix and Cyanotis arachnoidea. This compound has a wide range of pharmacological effects in the synthesis of substances such as proteins and nucleic acids, sugar metabolism, lipid component metabolism, central nervous system and bone development, etc., and has good prospects for medicinal use.

Liu Zhigang (Study on Therapeutic Material Basis of XieZhuo ChuBi Fang for Hyperuricemia [D]. Guangzhou University of Chinese Medicine, 2011.) conducted a study on the treatment of hyperuricemia by XieZhuo ChuBi Fang, in which the main herbal ingredients in XieZhuo ChuBi Fang are Smilacis Glabrae Rhizoma, Dioscoreae Spongiosae Rhizoma, Achyranthis Bidentatae Radix, Cremastrae Pseudobulbus Pleiones Pseudobulbus and vaccariae Semen, of which the main active ingredients of the monarch drug Smilacis Glabrae Rhizoma are flavonoids and Dioscoreopsis sapogenins; Achyranthis Bidentatae Radix, as the ministerial drug, mainly contains the active ingredients of triterpene sapogenins, ecdysterones, and inokosterone, etc. XieZhuo ChuBi Fang through the mutual compounding of the components, total flavonoids in the cell growth activity without significant inhibition of the conditions, the concentration of 1-2.5 g/L, the group containing ecdysterone on the absorption of uric acid has a significant inhibitory effect, but the active ingredient of this formula is not only ecdysterone, its total flavonoids, total sterols, saponins and alkaloids, lignans, synergistic formulations are also the prescription of the active ingredients.

Although there have been studies on uric acid-lowering drugs in the prior art, it is also crucial for the protection of kidney injury caused by hyperuricemia. Currently, no experimental studies and applications have been found related to the use of β-ecdysterone as the sole active component in the preparation of a medicine for uric acid-lowering or/and hyperuricemic kidney injury, and therefore, the present disclosure provides an application of β-ecdysterone and pharmaceutically acceptable salts thereof as the sole active component in preparation of a medicine for uric acid-lowering or hyperuricemic kidney injury.

### SUMMARY

The present disclosure addresses the problems of the prior art and provides an application of β-ecdysterone in preparation of a medicine for treating hyperuricemic kidney injury; the drug, β-ecdysterone or/and isomers thereof, or/and pharmaceutically acceptable salts, esters or/and prodrugs thereof are the sole active components in the drug, which provides a new drug for uric acid-lowering and/or hyperuricemic kidney injury, with the advantages of high safety, efficacy, well-defined composition, and controlled quality and dosage.

In order to realize the above purposes, the technical solutions adopted in the present disclosure are as follows:

Firstly, the present disclosure provides an application of β-ecdysterone in preparation of a medicine for uric acid-lowering and/or hyperuricemic kidney injury, the β-ecdysterone is β-ecdysterone or/and isomers thereof, or/and pharmaceutically acceptable salts, esters or/and prodrugs thereof; the molecular formula of the β-ecdysterone is C₂₇H₄₄O₇, and the chemical structure formula is shown as:

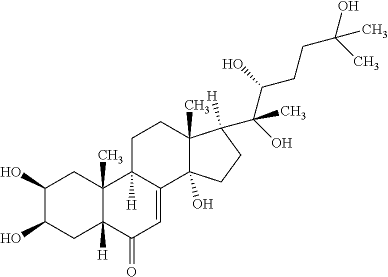

Preferably, in the application, the drug is a drug having the effect of reducing the blood uric acid level in hyperuricemia.

Preferably, in the application, the drug is a drug having the effect of improving kidney injury in hyperuricemia, reducing blood creatinine and urea nitrogen, and reducing urate crystals.

Preferably, in the application, the drug is a drug having the effect of reducing tubular dilatation degree and tubular wall thinning, and reducing the renal epithelial cell detachment.

Preferably, in the application, the drug comprises the sole active ingredient β-ecdysterone or/and isomers thereof, or/and pharmaceutically acceptable salts, esters or/and prodrugs thereof, and pharmaceutically acceptable carriers thereof.

In the present disclosure, the carriers, are all pharmaceutically acceptable carriers including, but not limited to, diluents, binders, absorbents, disintegrants, dispersants, wetting agents, co-solvents, buffers, surfactants.

In the present disclosure, the type and manufacturer of the carrier do not have a significant effect on the technical effect of the drug.

In the present disclosure, the carrier, may be starch, dextrin, sucrose, milk powder, sweeteners, mannitol, lactose, cellulosics and derivatives thereof, calcium carbonate, cyclodextrins, β-cyclodextrins, phospholipid materials, magnesium stearate, talc, essence.

Preferably, in the application, the mode of administration of the drug comprises oral, sublingual, oral mucosal, intravenously, intramuscularly, intraperitoneally, subcutaneously, transdermally, nasally and rectally routes.

In the present disclosure, in the application, the dosage form of the drug may be solid, liquid or gas.

Preferably, in the application, the dosage form of the drug includes powders, tablets, granules, pills, hard or soft capsules, creams, ointments, hard creams, gels, pastes, dispersions, patches, solutions, suspensions, injections, syrups, liniments, emulsions, tinctures, elixirs, aerosols, and sprays.

The present disclosure then provides a uric acid-lowering drug comprising an active ingredient and a pharmaceutically acceptable carrier thereof.

Preferably, the active ingredient is β-ecdysterone or/and isomers thereof, or/and pharmaceutically acceptable salts, esters or/and prodrugs thereof.

The present disclosure then provides a drug for treating hyperuricemic kidney injury, comprising an active ingredient and a pharmaceutically acceptable carrier thereof.

Preferably, the active ingredient is β-ecdysterone or/and isomers thereof, or/and pharmaceutically acceptable salts, esters or/and prodrugs thereof.

Further, the present disclosure provides a drug for uric acid-lowering and/or treating hyperuricemic kidney injury, comprising an active ingredient and a pharmaceutically acceptable carrier thereof.

Preferably, the active ingredient is β-ecdysterone or/and isomers thereof, or/and pharmaceutically acceptable salts, esters or/and prodrugs thereof.

Preferably, in the drug, the active ingredient is used in a dose of 20-100 mg/kg.

Further preferably, in the drug, the active ingredient is used in a dose of 25-50 mg/kg, more preferably 50 mg/kg.

Preferably, the dosage form of the drug includes powders, tablets, granules, pills, hard or soft capsules, creams, ointments, ointments, gels, pastes, dispersions, patches, solutions, suspensions, injections, syrups, liniments, emulsions, tinctures, elixirs, aerosols and sprays.

In relation to the prior art, the present disclosure has the following beneficial effects:

1, the application of β-ecdysterone in the present disclosure, β-ecdysterone can significantly reduce the blood uric acid level, showing a significant uric acid-lowering effect.

2, the application of β-ecdysterone in the present disclosure, β-ecdysterone improves kidney injury by reducing uric acid deposition in the kidney.

3, the application of β-ecdysterone in the present disclosure, β-ecdysterone can reduce blood creatinine and urea nitrogen, reduce tubular dilatation degree and tubular wall thinning, reduce renal epithelial cell detachment, and have a significant improvement effect on kidney injury.

4, the application of β-ecdysterone in the present disclosure, β-ecdysterone has both obvious uric acid-lowering effect and can reduce uric acid-induced kidney injury, with the advantages of precise efficacy, well-defined composition, controllable quality and dosage, and remarkable effect.

### BRIEF DESCRIPTION OF THE DRAWINGS

FIG. 1 shows the observation graph of the effect of administration of β-ecdysterone on urate deposition in rat kidney tissues, wherein A is the control group, B is the model group, C and D are both β-ecdysterone underdose group, E is the β-ecdysterone low dose group, F is the β-ecdysterone medium dose group, G is the β-ecdysterone high dose group, H is the β-ecdysterone overdose group, I is the allopurinol group, and J is the benzbromarone group.

FIG. 2 shows the pathological observation graph of rat kidney tissues, wherein A is the control group, B is the model group, C and D are both β-ecdysterone underdose group, E is the β-ecdysterone low dose group, F is the β-ecdysterone medium dose group, G is the β-ecdysterone high dose group, H is the β-ecdysterone overdose group, I is the allopurinol group, and J is the benzbromarone group.

### DETAILED DESCRIPTION OF THE EMBODIMENTS

The following non-limiting examples may enable those of ordinary skill in the art to more fully understand the present disclosure, but do not limit the present disclosure in any way. The following is only an exemplary illustration of the scope of protection claimed herein, and a person skilled in the art may make various changes and modifications to the present disclosure in accordance with the disclosed contents, which should also fall within the scope of protection claimed herein.

When the examples give ranges of values, it is to be understood that, unless otherwise indicated in the present disclosure, the two endpoints of each range of values and any of the values between the two endpoints are optional. Unless otherwise defined, all technical and scientific terms used herein have the same meaning as is commonly understood by one of ordinary skill in the art to which the present disclosure belongs.

The present disclosure is further described below by way of specific examples. The various chemical reagents used in the examples of the present disclosure are obtained by conventional commercial means if not otherwise specified.

In the present disclosure, the β-ecdysterone (CAS No.: 5289-74-7) is widely available from a wide range of sources, which can be procured commercially, or can be obtained by extraction from plants and animals; plant/animal sources that can be used for extraction include, but are not limited to, Achyranthis Bidentatae Radix, Cyanotis arachnoidea, shrimps, crabs, silkworms, etc. β-ecdysterone can also be obtained by methods such as semi-synthesis and/or total synthesis.

In the present disclosure, the molecular formula of the β-ecdysterone is C₂₇H₄₄O₇, and the chemical structure formula is shown as:

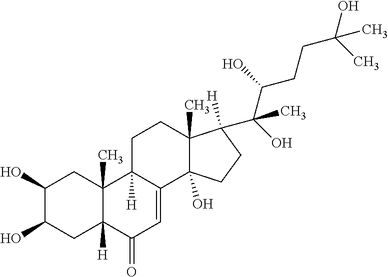

From ¹H NMR and ¹³C NMR characterization data, the structure of β-ecdysterone applied in the present disclosure was determined.

In the following examples, the β-ecdysterone is purchased from Baoji Herbest Bio-Tech Co., Ltd, with batch number HR20625W5; allopurinol is purchased from The PLACE Pharmaceutical (Jiangsu) Co., Ltd., with batch number 20190814; and benzbromarone is purchased from Aladdin, with batch number B124801. The active ingredients or drugs used in the present disclosure, whose products of different manufacturers do not have significant influence on the effect.

### Example 1 Effect of β-Ecdysterone on Blood Uric Acid Level in Rats with Hyperuricemia

### 1. Materials and Methods

### 1.1 Grouping of Animals and Mode of Administration

There were 80 Male SPF grade SD rats, weighing 200-220 g, were purchased from Beijing Vital River Laboratory Animal Technology Co., Ltd., license No. SCXK (Beijing) 2016-0006. The animals were housed in a barrier environment animal laboratory, with automatic temperature control (temperature 20-26° C.), relative humidity 40-70%, and full fresh air. Artificial light was adopted with a 12-hour light-dark cycle. Group experiments were started after 1 day of acclimatization. Animals were fed with standard rat pellet feed, provided by Beijing Keao Xieli Feed Co. Ltd., with purified water.

The rats were randomly divided into 10 groups of 8 rats each:

(1) negative control group (control group); (2) model group; (3) β-ecdysterone underdose group-1, 6 mg/kg; (4) β-ecdysterone underdose group-2, 12 mg/kg; (5) β-ecdysterone low dose group 25 mg/kg; (6) β-ecdysterone medium dose group 50 mg/kg; (7) β-ecdysterone high dose group 100 mg/kg; (8) β-ecdysterone overdose group 200 mg/kg; (9) allopurinol group 25 mg/kg; (10) benzbromarone group 25 mg/kg.

1.2 Establishment of Adenine Combined with Ethambutol-Induced Hyperuricemia Combined with Kidney Injury Model in Rats

Adenine was purchased from Sigma-Aldrich (USA), and 0.5% CMC-Na (sodium carboxymethyl cellulose) solution was used to formulate a suspension at a concentration of 20 mg/mL, ready-to-use, with an administration volume of 5 mL/kg and a dosage of 100 mg/kg. Ethambutol hydrochloride tablets were purchased from Chengdu Jinhua Pharmaceutical Co., Ltd, and 0.5% CMC-Na solution was used to formulate a suspension at a concentration of 50 mg/ml, ready-to-use, with an administration volume of 5 mL/kg and a dosage of 250 mg/kg. The modeling drug adenine 100 mg/kg and ethambutol 250 mg/kg were administered by gavage in the model group and the CQ group at each dose, from day 1, and both were administered once a day for a total of 7 days, to prepare a hyperuricemia rat model. An equal amount of 0.5% CMC-Na solution was administered by gavage in the control group at the relative time.

### 1.3 Sample Processing and Statistical Methods

Observation and detection indicators: fasting for 16 hours after the last administration, blood was collected from the abdominal aorta of rats in each group, centrifuged at 3500 rpm for 15 minutes after standing, separated and collected serum, and the content of uric acid in the serum was measured using an Automatic Chemical Analysis.

Statistical methods: data were expressed as mean±standard deviation and analyzed by one-way ANOVA. Each administration group was compared with the model group separately.

### 2. Results

The detection results of the effect of β-ecdysterone on blood uric acid levels in rats with hyperuricemia are shown in Table 1:

TABLE 1

| | | | Uric acid |
|---|---|---|---|
| | Dosages | Blood uric acid | reduction |
| Groups | (mg/kg) | (μmol/L) | Percentage (%) |
| Control group | — | 115.03 ± 16.48* | / |
| Model group | — | 162.46 ± 41.17 | / |
| β-ecdysterone | 6 | 154.76 ± 38.71 | 4.98 |
| underdose group-1 | | | |
| β-ecdysterone | 12 | 147.55 ± 39.28 | 10.10 |
| underdose group-2 | | | |
| β-ecdysterone low dose | 25 | 117.91 ± 43.74 | 37.78 |
| group | | | |
| β-ecdysterone medium | 50 | 110.83 ± 25.02* | 46.58 |
| dose group | | | |
| β-ecdysterone high dose | 100 | 120.19 ± 68.99 | 35.17 |
| group | | | |
| β-ecdysterone overdose | 200 | 127.56 ± 61.77 | 27.36 |
| group | | | |
| Allopurinol group | 25 | 109.11 ± 21.66* | 48.90 |
| Benzbromarone group | 25 | 112.53 ± 30.78* | 44.37 |

Compared with the model group,
*p < 0.05.

The results showed that the serum uric acid level in the model group was significantly higher (p<0.05) compared to the control group, and the administration of a medium dose of β-ecdysterone had a significant reducing effect on the blood uric acid level of the animals with hyperuricemia, with a reduction percentage of 46.58% (p<0.05). The results suggested that the active ingredient in the present disclosure at 50 mg/kg, β-ecdysterone, had a significant effect of reducing blood uric acid. However, when the dose of β-ecdysterone was too low, the blood uric acid level was not significantly reduced compared to the model group. In addition, the positive drugs allopurinol and benzbromarone showed significant reducing effect on the blood uric acid level of the animals with hyperuricemia, with a reduction percentage of 48.90% and 44.37% respectively, which was similar to the uric acid-lowering effect of the β-ecdysterone group at 50 mg/kg.

### Example 2 Improvement Effect of β-Ecdysterone on Kidney Injury in Rats with Hyperuricemia

### 1. Materials and Methods

The establishment of the model as well as the experimental grouping and administration were the same as in Example 1.

1.1 Kidney function biochemical indicators detection: the method of blood sampling and serum collection was the same as in Example 1, and an Automatic Chemical Analysis was used to detect the content of serum creatinine (SCr) by using the creatine oxidase method, and to detect the content of blood urea nitrogen (BUN) by using the urease method and the kinetic method. the effect of β-ecdysterone on the level of blood BUN and SCr of rats with hyperuricemia is shown in Table 2.

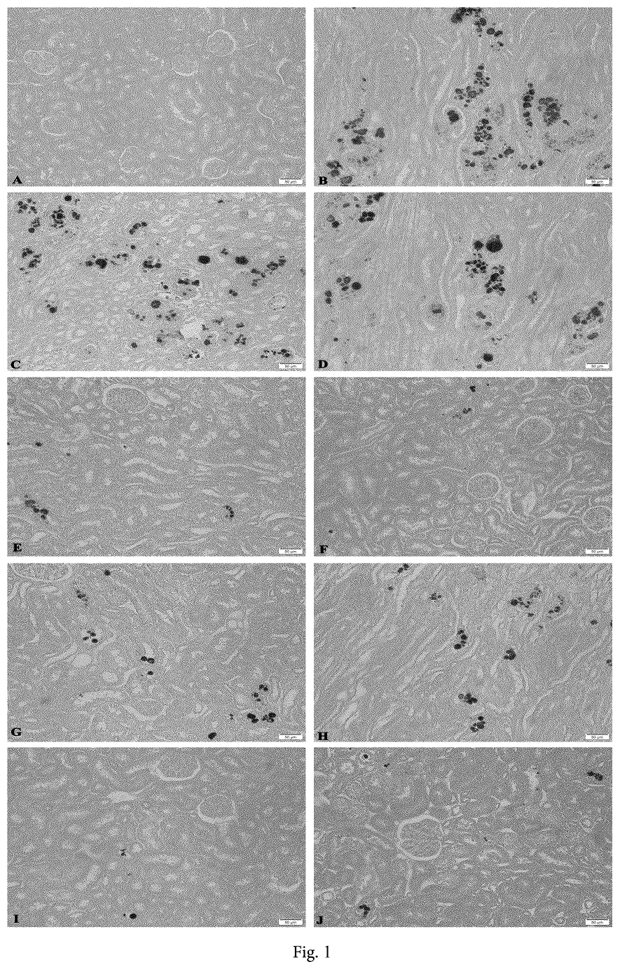

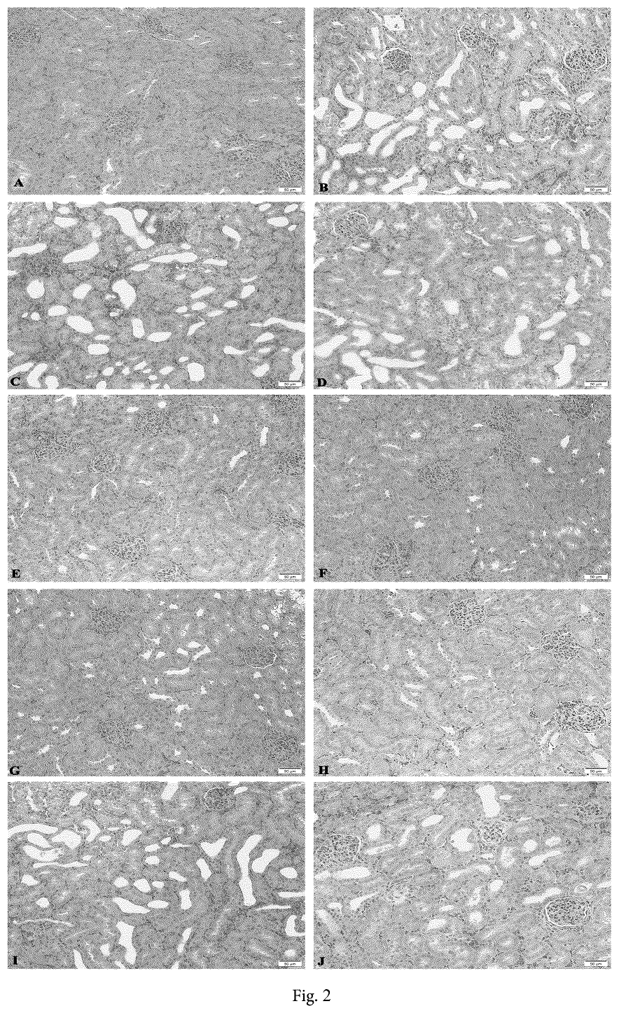

1.2 Pathological examination of kidney tissue: anesthetize rats, dissect, and remove kidney tissue. The kidney tissues were fixed with 10% neutral formalin. After sufficient fixation, sampling, gradient dehydration, xylene transparency, paraffin embedding, and preparation of 3 μm paraffin sections were performed according to routine procedures. The kidney tissue lesions as well as urate deposition in the kidney tissues were examined by light microscopy using, HE (hematoxylin & eosin) staining and hexamine silver staining, respectively. The detection results are shown in FIG. 1-FIG. 2. FIG. 1 shows the observation graph of the effect of β-ecdysterone administration on urate deposition in rat kidney tissues (hexamine silver staining), and FIG. 2 shows the observation graph of pathology of rat kidney tissues. Among them, A in FIG. 1 and FIG. 2 is the control group, B is the model group, C and D are both β-ecdysterone underdose group, E is β-ecdysterone low dose group, F is β-ecdysterone medium dose group, G is β-ecdysterone high dose group, H is β-ecdysterone overdose group, I is allopurinol group, and J is benzbromarone group. The length unit in the lower right corner of the figure is 50 μm.

### 2. Results

### 2.1 Effect of β-Ecdysterone on Urate Deposition in Rat Kidney Tissues

Urate crystals deposition was not observed in the control group, and a large number of urate crystals deposition was observed in the kidney tissue in the model group. The deposited urate crystals were significantly reduced after the administration of β-ecdysterone. The results indicated that β-ecdysterone could significantly reduce the urate crystals deposition in the kidney tissues of rats with hyperuricemia, with the most obvious effect in the 50 mg/kg group. While no significant reduction of urate crystals was seen when the dose of β-ecdysterone was lower. In addition, the deposition of urate crystals was also significantly reduced after administration of the positive drugs allopurinol and benzbromarone.

### 2.2 Effects of β-Ecdysterone on Biochemical Indicators Related to Kidney Injury in Rats

The results indicated that both serum BUN and SCr levels were significantly higher in the model group compared to the control group (p<0.001), suggesting that the rats with hyperuricemia exhibited some kidney injury. The serum BUN and SCr levels were significantly reduced (p<0.01) after the administration of β-ecdysterone compared with that of the model group, indicating that β-ecdysterone had a significant improvement effect on kidney injury caused by hyperuricemia. However, when the dose of β-ecdysterone was too high, only a slight decreasing trend was visible in serum BUN and SCr levels compared with the model group, and when the dose of β-ecdysterone was too low, no significant difference was seen in serum BUN and SCr levels compared with the model group. In addition, the serum BUN and SCr levels of the positive drugs allopurinol and benzbromarone did not show significant differences compared with the model group, suggesting that allopurinol and benzbromarone did not have a significant improvement effect on kidney injury caused by hyperuricemia.

### 2.3 Effect of β-Ecdysterone on the Pathology of Rat Kidney Tissues

In the control group, the tissue structures of renal interstitium, renal tubules and glomerular were basically normal, the cells were neatly arranged, and no pathological changes such as tubular dilatation, degeneration or necrosis were observed.

In the model group, the glomerular morphology was basically normal in the renal tissues, but pathological changes such as tubular wall thinning, severe dilatation, epithelial cell detachment, eosinophilic degeneration and inflammatory cell infiltration were observed.

In the β-ecdysterone underdose group, the glomerular morphology was basically normal, but pathological changes such as severe tubular expansion, epithelial cell detachment, eosinophilic degeneration and inflammatory cell infiltration could still be observed.

In the β-ecdysterone low dose group, the glomerular morphology was basically normal in the kidney tissues, and the phenomenon of tubular dilatation was reduced compared with that of the model group, but a small amount of tubular dilatation could still be observed, and pathological changes such as tubular epithelial cell detachment could be observed locally.

In the β-ecdysterone medium dose group, the glomerular morphology was basically normal in the kidney tissues, and the phenomenon of tubular dilatation was significantly reduced, with only occasional tubular epithelial cell detachment.

In the β-ecdysterone high dose group, the glomerular morphology was basically normal in the kidney tissues, and the phenomenon of tubular dilatation was reduced compared with that of the model group, but a small amount of tubular dilatation could still be observed locally, and the improvement effect was inferior to that of the medium dose group.

In the β-ecdysterone overdose group, the glomerular morphology was basically normal in the kidney tissues, but pathological changes such as mild tubular dilatation and tubular epithelial cell detachment, and tubular wall thinning in some of the tubules were observed locally.

In the allopurinol and benzbromarone groups, the glomerular morphology was basically normal in the kidney tissues, but pathological changes such as severe renal tubules dilatation, tubular wall thinning, eosinophilic degeneration, and inflammatory cell infiltration were observed.

The combination of hexamine silver staining results, biochemical results and pathological results suggested that the active ingredient in the present disclosure, β-ecdysterone, may have a significant protective effect on kidney injury combined with hyperuricemia by decreasing uric acid deposition in the kidneys, and the effect of the 50 mg/kg group is the most obvious. While the positive drugs allopurinol and benzbromarone only saw a better reduction of uric acid deposition in the kidneys, but did not have a significant protective effect on the kidney injury combined with hyperuricemia.

TABLE 2

| | Dosages | | |
|---|---|---|---|
| Groups | (mg/kg) | BUN (mmol/L) | SCr (μmol/L) |
| Control group | — | 4.28 ± 0.46*** | 29.79 ± 4.92*** |
| Model group | — | 11.01 ± 1.82 | 50.60 ± 8.25 |
| β-ecdysterone | 6 | 11.34 ± 4.11 | 51.72 ± 7.35 |
| underdose group-1 | | | |
| β-ecdysterone | 12 | 9.81 ± 3.76 | 47.56 ± 6.11 |
| underdose group-2 | | | |
| β-ecdysterone low | 25 | 7.36 ± 1.71** | 40.73 ± 5.79* |
| dose group | | | |
| β-ecdysterone | 50 | 7.19 ± 1.00** | 41.30 ± 4.08* |
| medium dose group | | | |
| β-ecdysterone high | 100 | 7.84 ± 2.04* | 42.15 ± 5.01 |
| dose group | | | |
| β-ecdysterone | 200 | 8.04 ± 3.96 | 44.30 ± 5.21 |
| overdose group | | | |
| Allopurine group | 25 | 12.17 ± 3.03 | 48.16 ± 6.95 |
| Benzbromarone | 25 | 10.91 ± 2.66 | 50.64 ± 7.92 |
| group | | | |

Compared with the model group,
*p < 0.05,
**p < 0.01,
***p < 0.001.

Finally, it should be noted that the above content is only used to illustrate the technical solution of the present disclosure, not a limitation on the scope of protection of the present disclosure, and simple modifications or equivalent replacements to the technical solution of the present disclosure by people of ordinary skill in the field do not depart from the substance and scope of the technical solution of the present disclosure.

What is claimed is:

1. An application of β-ecdysterone in preparation of a medicine for uric acid-lowering and/or hyperuricemic kidney injury, wherein the β-ecdysterone is β-ecdysterone or/and isomers thereof, or/and pharmaceutically acceptable salts, esters or/and prodrugs thereof; the molecular formula of the β-ecdysterone is C₂₇H₄₄O₇, and the chemical structure formula is shown as:

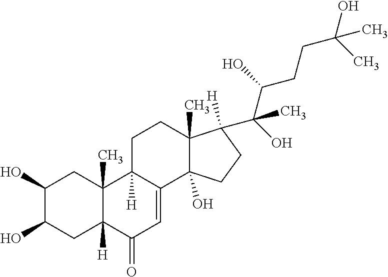

2. The application according to claim 1, wherein the drug is a drug having the effect of reducing the blood uric acid level in hyperuricemia.

3. The application according to claim 1, wherein the drug is a drug having the effect of improving kidney injury in hyperuricemia, reducing blood creatinine and urea nitrogen, and reducing urate crystals.

4. The application according to claim 1, wherein the drug is a drug having the effect of reducing tubular dilatation degree and tubular wall thinning, and reducing the renal epithelial cell detachment.

5. The application according to claim 1, wherein the drug comprises the sole active ingredient β-ecdysterone or/and isomers thereof, or/and pharmaceutically acceptable salts, esters or/and prodrugs thereof, and pharmaceutically acceptable carriers thereof.

6. The application according to claim 1, wherein the dosage form of the drug is solid, liquid or gas.

7. The application according to claim 6, wherein the dosage form of the drug is selected from one of the following: powders, tablets, granules, pills, hard capsules, soft capsules, creams, ointments, hard creams, gels, pastes, dispersions, patches, solutions, suspensions, injections, syrups, liniments, emulsions, tinctures, elixirs, aerosols, and sprays.

8. A uric acid-lowering drug, wherein comprising an active ingredient and a pharmaceutically acceptable carrier thereof; the active ingredient is β-ecdysterone or/and isomers thereof, or/and pharmaceutically acceptable salts, esters or/and prodrugs thereof; the molecular formula and chemical structure formula of the β-ecdysterone are shown in claim 1.

9. The drug according to claim 8, wherein the active ingredient is used in a dose of 20-100 mg/kg.

10. A drug for treating hyperuricemic kidney injury, wherein comprising an active ingredient and a pharmaceutically acceptable carrier thereof; the active ingredient is β-ecdysterone or/and isomers thereof, or/and pharmaceutically acceptable salts, esters or/and prodrugs thereof; the molecular formula and chemical structure formula of the β-ecdysterone are shown in claim 1.

11. The drug according to claim 10, wherein the active ingredient is used in a dose of 20-100 mg/kg.

12. A drug for uric acid-lowering and/or treating hyperuricemic kidney injury, wherein comprising an active ingredient and a pharmaceutically acceptable carrier thereof; the active ingredient is β-ecdysterone or/and isomers thereof, or/and pharmaceutically acceptable salts, esters or/and prodrugs thereof; the molecular formula and chemical structure formula of the β-ecdysterone are shown in claim 1.

13. The drug according to claim 12, wherein the active ingredient is used in a dose of 20-100 mg/kg.
